(19) United States
(12) Patent Application Publication
(10) Pub. No.: US 2018/0256108 A1
(43) Pub. Date: Sep. 13, 2018

(54) INSERTER AND METHOD OF INSERTING AN IMPLANT UNDER THE SKIN

(71) Applicant: Profusa, Inc., South San Francisco, CA (US)

(72) Inventors: Kit Yee Au-Yeung, San Francisco, CA (US); Ben Hwang, Lafayette, CA (US); William A. McMillan, La Honda, CA (US); Kristopher Konawalik, San Francisco, CA (US); Jeffrey Servaites, Oakland, CA (US); Sandhya Santhanaraman, Sunnyvale, CA (US)

(21) Appl. No.: 15/920,061

(22) Filed: Mar. 13, 2018

Related U.S. Application Data
(60) Provisional application No. 62/470,712, filed on Mar. 13, 2017.

(51) Int. Cl. A61B 5/00

(52) U.S. Cl. CPC A61B 5/6846; A61B 2560/063

(57) ABSTRACT

Some embodiments described herein relate to an apparatus, such as an inserter, having a housing and a needle at least partially disposed within the housing. An actuator can be coupled to the needle and configured to move the needle between an actuated configuration and a retracted configuration. A wire can be at least partially disposed within the needle. The wire can be fixedly and immovably coupled to the housing. In some embodiments a biosensor can be disposed within the needle and the apparatus can be configured to deliver or implant the biosensor to a patient, for example, under the patient's skin. The biosensor can constructed of a non-rigid material, such as hydrogel. In some embodiments, the apparatus can be configured to deliver or implant the biosensor without applying a force to the biosensor.

### CROSS-REFERENCE TO RELATED APPLICATIONS

This application claims the benefit of U.S. Provisional Patent App. No. 62/470,712 entitled “An Applicator and Method for Inserting a Material Under the Skin of a Human,” filed on Mar. 13, 2017, the entire disclosure of which is incorporated herein by reference.

### TECHNICAL FIELD

The presently disclosed subject matter relates generally to technology for implanting material under the skin and more particularly to an inserter for and method of inserting non-rigid biosensors under the skin.

### BACKGROUND

Fully implantable biosensors for use in medical applications have significant promise in diagnosing and managing human diseases. A biosensor is any device operable to detect a biological parameter (e.g., heart rate, respiration rate, etc.) and/or a quantity/concentration of a biological analyte (e.g., glucose, lactate, molecular oxygen, glycerol, glutamate, hydrogen peroxide, etc.), and emits a detectable signal (e.g., optical, electrical, radio), frequently to a secondary device for storage, analysis, and/or human-readable presentation. An implantable biosensor can be implanted within the subcutaneous tissue space as well as within the layers of skin, intramuscularly or within the vasculature. Implanting the biosensor into these locations permits the sensing of biological parameters and/or biological analytes for both discrete and/or continuous monitoring.

The implantation process of such a miniaturized biosensors can be accomplished by injection through a conventional, medical-grade needle/syringe. However, there can be certain drawbacks to current implantation processes. For example, some known methods involve placing a miniaturized biosensor in the tip of the needle for injection. In such a method, however, it may be difficult to deploy the biosensor while withdrawing the needle. Namely, when withdrawing the needle, sometimes the biosensor will remain in the tip of the needle rather than ejecting and being left behind in the tissue. Therefore, new approaches are needed to ensure reliable deployment of materials, such as biosensors, under the skin.

### SUMMARY

Some embodiments described herein relate to an apparatus, such as an inserter, having a housing and a needle at least partially disposed within the housing. An actuator can be coupled to the needle and configured to move the needle between an actuated configuration and a retracted configuration. A wire can be at least partially disposed within the needle. The wire can be fixedly coupled to the housing. In some embodiments, a biosensor can be disposed within the needle and the apparatus can be configured to deliver or implant the biosensor to a patient, for example, under the patient's skin. The biosensor can constructed of a non-rigid material, such as hydrogel. In some embodiments, the apparatus can be configured to deliver or implant the biosensor without applying a force to the biosensor.

### BRIEF DESCRIPTION OF THE DRAWINGS

Having thus described the presently disclosed subject matter in general terms, reference will now be made to the accompanying Drawings, which are not necessarily drawn to scale, and wherein:

FIG. 1 is perspective view of an inserter configured to insert an implant under the skin, according to an embodiment.

FIG. 2A is a top view, FIG. 2B is a side view, and FIG. 2C is an end view of the inserter of FIG. 1.

FIG. 3 is an exploded view of the inserter of FIG. 1.

FIG. 4 is a perspective view of the inserter of FIG. 1, with a cap removed.

FIG. 5A is a top view, FIG. 5B is a side view, and FIG. 5C is end view of the inserter of FIG. 4.

FIG. 6A and FIG. 6B are perspective views showing the needle of an inserter, according to an embodiment, in a fully actuated and fully retracted configuration, respectively.

FIG. 7A and FIG. 7B illustrate loading an implant into the needle of an inserter, according to an embodiment.

FIG. 8A through FIG. 8F illustrate an example of using an inserter to insert an implant under the skin.

FIG. 9A through FIG. 9E illustrate an example of a process of using a wire of an inserter to assist deploying the implant under the skin.

FIG. 10 is a flow diagram of a method of using an inserter to insert an implant under the skin, according to an embodiment.

### DETAILED DESCRIPTION

The disclosed subject matter now will be described more fully with reference to the accompanying drawings. Like numbers refer to like elements throughout. The disclosed subject matter may be embodied in many different forms and should not be construed as limited to the embodiments set forth herein; rather, these embodiments are provided so that this disclosure will satisfy applicable legal requirements. Indeed, many modifications and other embodiments of the disclosed subject matter set forth herein will come to mind to one skilled in the art to which the disclosed subject matter pertains having the benefit of the teachings presented in the foregoing descriptions and the associated drawings. Therefore, it is to be understood that the disclosed subject matter is not to be limited to the specific embodiments disclosed and that modifications and other embodiments are intended to be included within the scope of the appended claims.

Some embodiments relate to an apparatus, such as an inserter, having a housing and a needle at least partially disposed within the housing. An actuator can be coupled to the needle and configured to move the needle between an actuated configuration and a retracted configuration. A wire can be at least partially disposed within the needle. The wire can be fixedly and/or immovably coupled to the housing. In some embodiments, an implant, such as a biosensor can be disposed within the needle and the apparatus can be configured to deliver the implant into a patient, for example, under the patient's skin. The implant can constructed of a non-rigid material, such as hydrogel. In some embodiments, the apparatus can be configured to deliver the implant without applying a force to the biosensor.

Some embodiments relate to an inserter that includes a housing that contains a hypodermic needle and a wire inside the needle. The needle may be moveable in relation to a stationary wire, the wire may be moveable in relation to a stationary needle, or both the needle and the wire are moveable. An actuator is provided for moving the needle, the wire, or both the needle and the wire.

Some embodiments described herein relate to a method that includes inserting a needle that contains an implant into a patient. The needle is inserted into the patient by moving a body of an inserter. After the needle is inserted into the patient, the needle can be moved relative to the inserter such that the implant is ejected from the needle. A wire partially disposed within the needle can cause the implant to remain stationary relative to the body of the inserter while the needle is moved. The needle can then be withdrawn from the patient by moving the body of the inserter, leaving the implant within the patient.

Some embodiments describe an implant to be inserted into a patient. The implant may be a rod-like (e.g., cylindrical), non-rigid, non-liquid material, such as, but not limited to, a polymer- and/or hydrogel-based biosensor. Inserting non-rigid implants poses significant challenges not present when inserting solids or liquids. When a solid is inserted into a body, force can be applied to the solid, for example by a plunger, to expel it from a needle. Similarly, liquids are virtually incompressible, so when a liquid is inserted, force applied to the liquid by a plunger will increase the pressure in the liquid causing the liquid to be expelled from the needle without altering the liquid. When a non-rigid implant is inserted, however, applying a force to insert the implant can cause the implant to deform or compress and/or damage the structure of the implant. Some embodiments relate to inserting a non-rigid implant into a body without applying a force directly to the implant. The non-rigid implant may be porous, and applying a force to the non-rigid implant can cause the pores to collapse, potentially permanently damaging the structure of the implant.

The implant can be loaded into a sharp tip of the needle, wherein the wire acts a stop against which the implant rests inside the needle. In one embodiment, the needle is retractable via the actuator relative to the stationary wire such that the motion of the needle, rather than applying a force to the implant, causes the implant to be ejected and/or deployed under the skin of a subject.

Inserters and methods described herein can be used to ensure reliable deployment of non-rigid implants, such as polymer- and/or hydrogel-based biosensors, under the skin. Additionally, the inserter and method can be used to ensure reliable deployment of hard rigid implants under the skin of the subject.

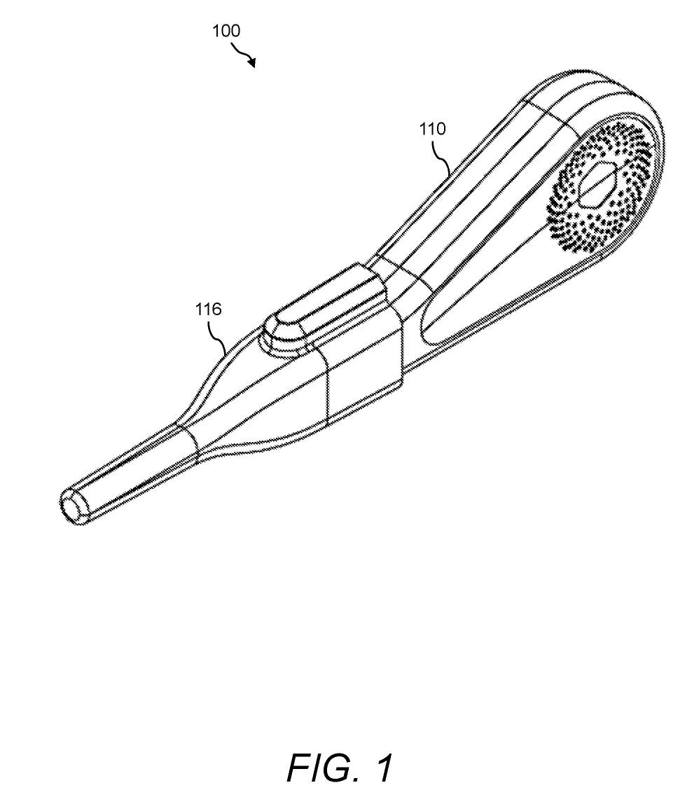

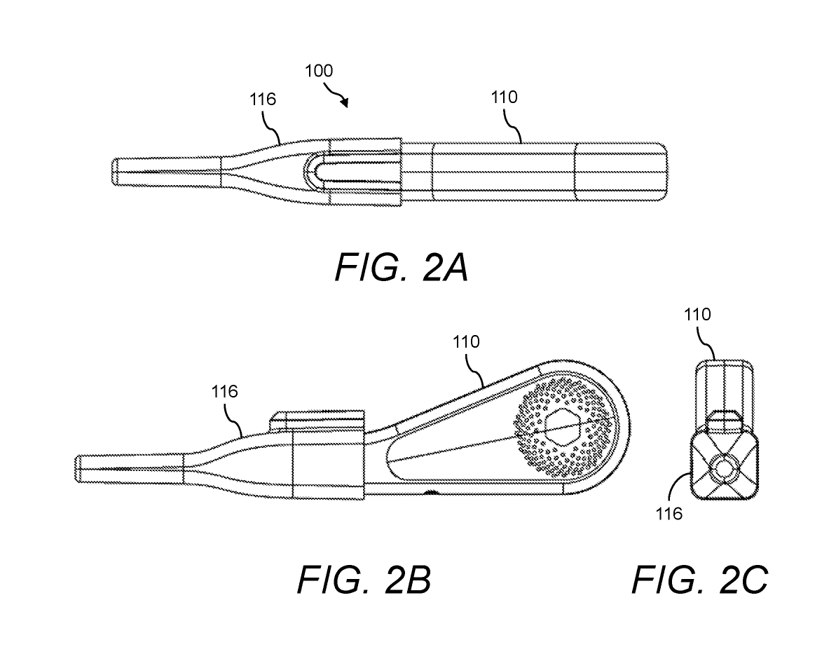

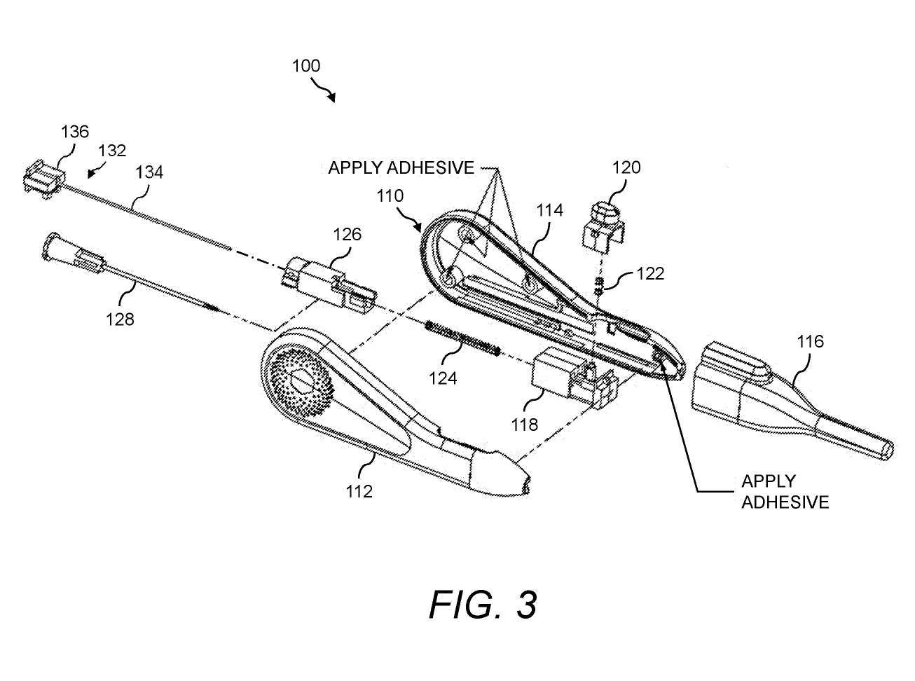

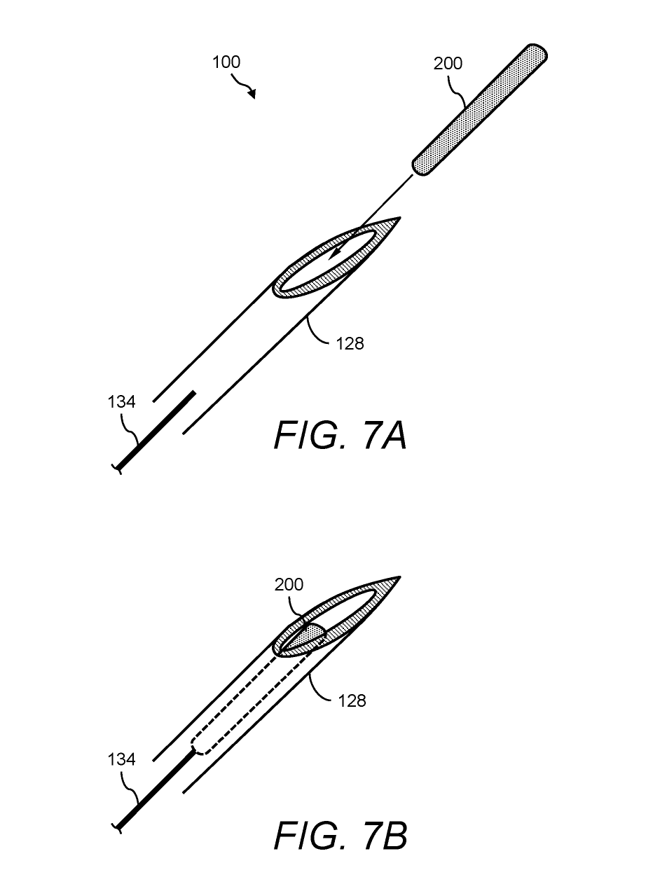

FIG. 1 is a perspective view of an inserter 100 for inserting an implant under the skin, according to an embodiment. FIG. 2A is a top view, FIG. 2B is a side view, and FIG. 2C is end view of the inserter 100. FIG. 3 is an exploded view of the inserter 100. The inserter 100 is an injector or injection pen for deploying a material (e.g., a biosensor) under the skin of a subject (e.g., a human subject, also called the patient). The inserter 100 is particularly suitable for deploying a non-rigid implant, although it can also be used to deploy a rigid solid implant or a liquid. More details of an example of the implant are shown and described herein below with reference to FIG. 7A and FIG. 7B.

The inserter 100 includes a housing 110, wherein the housing 110 is a two-piece housing that includes a first housing side 112 and a second housing side 114. The inserter 100 also includes a cap 116 for fitting on the distal end of the housing 110. The inserter 100 further includes, a shuttle 118, an actuator 120, an actuator spring 122, a spring extension 124, an inject shroud 126, a needle 128, and a plunger assembly 132. The plunger assembly 132 includes a wire 134 and a plunger block 136. The first housing side 112, the second housing side 114, the cap 116, the shuttle 118, the actuator 120, the actuator spring 122, the spring extension 124, the inject shroud 126, the needle 128, the plunger assembly 132, the wire 134, and the plunger block 136 are arranged, for example, as shown in FIG. 3.

The first housing side 112 and the second housing side 114 of the housing 110, the cap 116, the shuttle 118, the actuator 120, the inject shroud 126, and the plunger block 136 can be formed, for example, of polycarbonate (PC) plastics (e.g., molded plastic). Further, the wire 134, the actuator spring 122, the spring extension 124, and the needle 128 can be formed, for example, of stainless steel. When assembled, first housing side 112 and second housing side 114 can be held together, for example, via an adhesive.

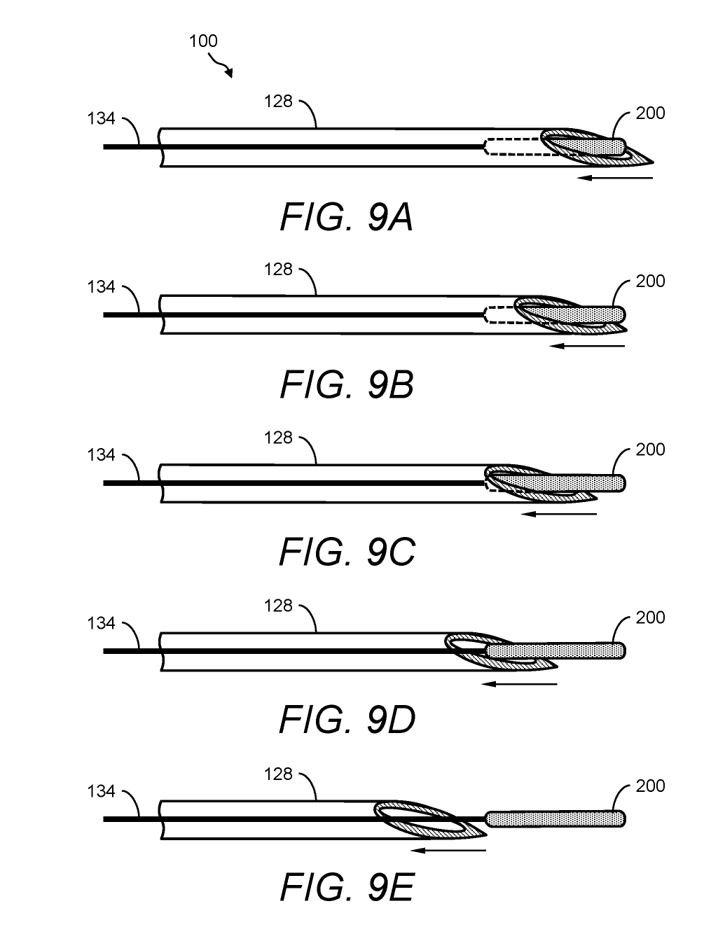

In the inserter 100, the wire 134 is inserted into the proximal end of the needle 128 and extends toward the distal tip of the needle 128 in which the implant is installed (see FIG. 9A through FIG. 9E). The distal tip of the needle 128 is the sharp end of the needle 128 that is inserted into the body.

As shown, the actuator 120 moves the needle 128 only and does not move the wire 134. Similarly stated, the wire 134 can be fixedly coupled to the housing 110 via the plunger block 136, such that the wire 134 is held immobile relative to the housing. The needle 128, spring extension 124, shuttle 118, and inject shroud 126 can define a passageway through which the wire 134 is inserted such that the needle 128, spring extension 124, shuttle 118, and inject shroud 126 can slide over and relative to the wire 134.

In another embodiment, the actuator 120 can be operable to move the wire 134 relative to the housing 110 and does not move the needle 128. In yet another example, the actuator 120 moves both the wire 134 and the needle 128.

As discussed in further detail herein, in some embodiments, the actuator 120 and/or the shuttle 118 can be operable to lock the needle 128 in a fully actuated and/or a fully retracted configuration. For example, the actuator spring 122 can urge the actuator 120 into one or more groves or recesses defined by the housing 110. When the actuator 120 is disposed in a recess, the housing 110 can resist or prevent the actuator 120 (and shuttle 118 and needle 128) from sliding relative to the housing 110. When the actuator 120 is depressed (e.g., by a user's thumb or finger) the actuator 120 (and shuttle 118 and needle 128) can slide relative to the housing 110, allowing the needle 128 to move between the fully actuated and fully retracted configurations. In some embodiments, the actuator 120 and the housing 110 can collectively lock the needle 128 into the fully actuated configuration. The spring extension 124 can urge the shuttle 118, actuator 120, and needle 128 into the fully actuated position, such that if the actuator 120 is released, the needle 128 can return to the fully actuated position. In other embodiments, the actuator 120 and the housing 110 can collectively lock the needle 128 into either or both of the fully retracted and the fully actuated position.

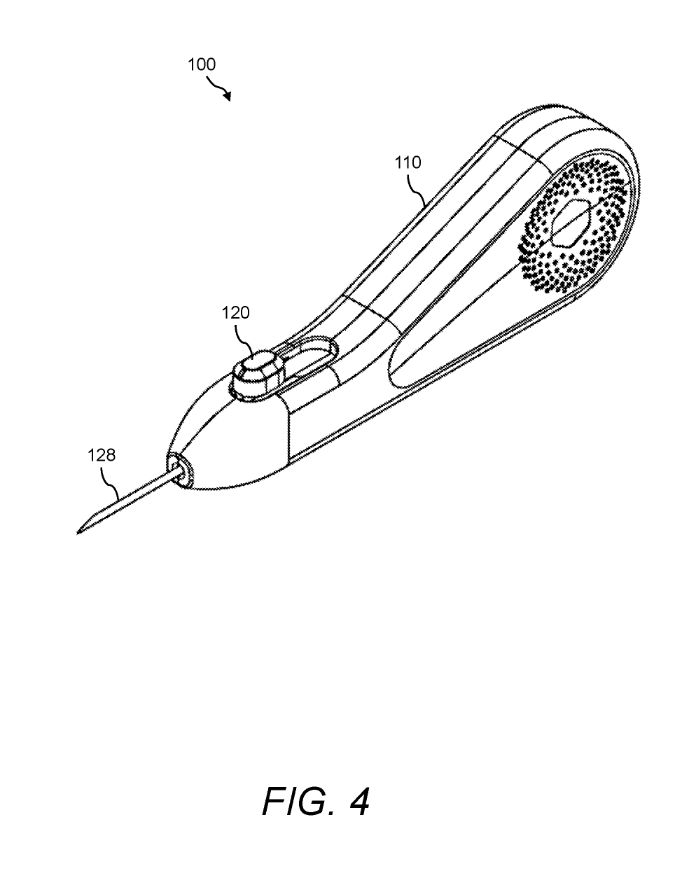

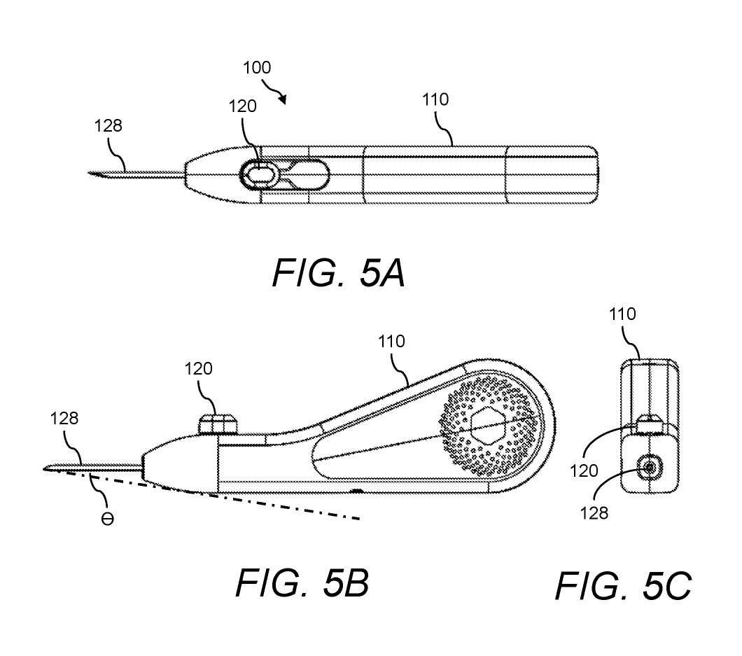

FIG. 4 is a perspective view of the inserter 100 absent the cap 116. The cap 116 can be removably and/or reversibly coupled to the housing 110. The cap 116 can function as a needle guard and prevent the needle 128 from contacting or puncturing a person or object 116 while the cap 116 is coupled to the housing 110. When the cap 116 is removed, as shown in FIG. 4, the needle 128 can be exposed. FIG. 5A is a top view, FIG. 5B is a side view, and FIG. 5C an end view of the inserter 100 absent the cap 116.

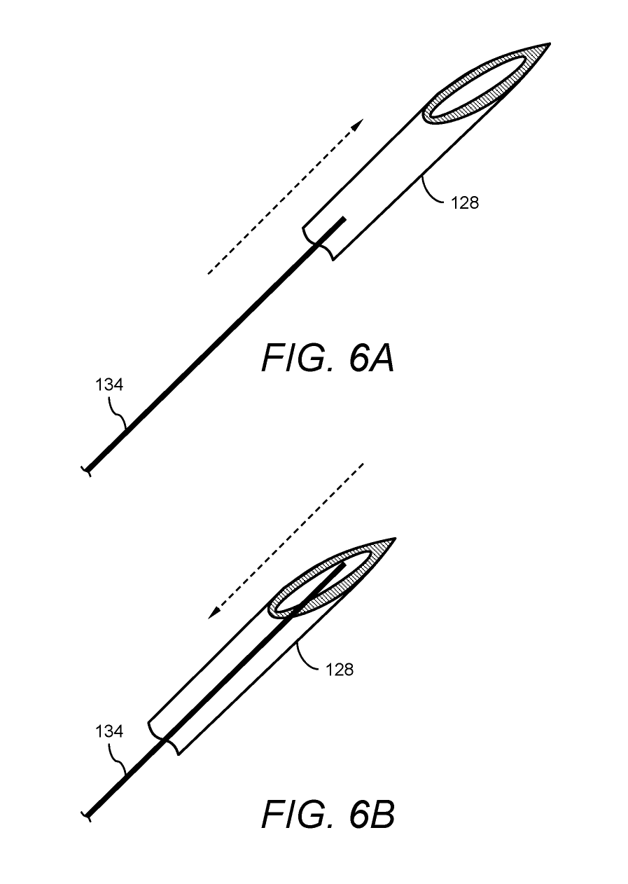

FIG. 6A and FIG. 6B are perspective views showing the needle 128 of the inserter 100 fully actuated and fully retracted, respectively, according to an embodiment. FIG. 6A illustrates the needle 128 in an extended configuration such that an empty space suitable for containing an implant is created within a distal portion of the needle 128. FIG. 6B illustrates needle 128 in a retracted configuration. As discussed above, the wire 134 is held stationary relative to the housing 110, such that when the needle 128 is retracted the distal end of the needle 128 moves closer to the distal end of the wire 128.

FIG. 7A and FIG. 7B are perspective views of an example of loading an implant 200 into the needle 128 of inserter 100. For example, the implant 200 may be a rod-like, non-rigid, non-liquid implant, such as a gel-like implant. In some embodiments, the implant 200 may be compressible and/or may have an internal structure that can be damaged by applying a force to one end. In this example, implant 200 has an elongated shape that is sized to be fitted into the sharp end of the needle 128. FIG. 7A shows the implant 200 aligned with the tip of the needle 128 and ready to load. FIG. 7B shows the implant 200 fully loaded into the tip of the needle 128. The distal end of the wire 134 acts as a stop against which the implant 200 rests.

In one example, the implant 200 may be a polymer- and/or hydrogel-based biosensor. Examples of hydrogel-based biosensors include, but are not limited to, the biosensors described with reference to U.S. Patent Pub. No. 2012/0265034, entitled “Tissue-Integrating Biosensors,” published on Oct. 18, 2012; and U.S. Pat. No. 9,375,494, entitled “Oxygen sensors,” issued on Jun. 28, 2016; the entire disclosures of which are incorporated herein by reference.

In one example, the inserter 100 may be operated with one hand. The inserter 100 may be about 12 cm in length. The needle 128 may be a commercially available stainless steel hypodermic needle. For example, the needle 128 may have a diameter of about 0.8 mm and length of about 20 mm.

The wire 134 serves to hold the implant 200 in place during the removal of the needle 128 in the channel created by the needle 128. The wire 134 may occupy a portion of the needle 128 and the implant 200 may occupy the remaining length of the needle 128. The wire 134 may occupy the proximal end of the needle 128 and the implant 200 may occupy the distal end of the needle 128. Together, the wire 134 and the implant 200 may occupy from about 80% to about 100% of the length of the needle 128 in one example, from about 90% to about 100% of the length of the needle 128 in another example, or from about 95% to about 100% of the length of the needle 128 in yet another example.

The wire 134 may occupy from about 1% to about 99% the length of the needle 128 in one example, from about from about 30% to about 95% of the length of the needle 128 in another example, from about 40% to about 90% of the length of the needle 128 in yet another example, from about 50% to about 80% of the length of the needle 128 in yet another example, or from about 70% to about 80% of the length of the needle 128 in still another example.

The implant 200 may occupy from about 1% to about 99% the length of the needle 128 in one example, from about 5% to about 70% of the length of the needle 128 in another example, from about 10% to about 60% of the length of the needle 128 in yet another example, from about 20% to about 50% of the length of the needle 128 in yet another example, or from about 20% to about 30% of the length of the needle 128 in still another example.

The implant 200 may be, for example, rod shaped or cylindrical shaped. For example, the implant 200 may be a rod-shaped, hydrogel-containing material that is about 5 mm long and about 0.5 mm in diameter. Generally, the implant 200 may be from about 0.1 mm to about 20 mm long in one example, from about 0.5 mm to about 10 mm long in another example, from about 1 mm to about 8 mm long in yet another example, or from about 2 mm to about 7 mm long in still another example.

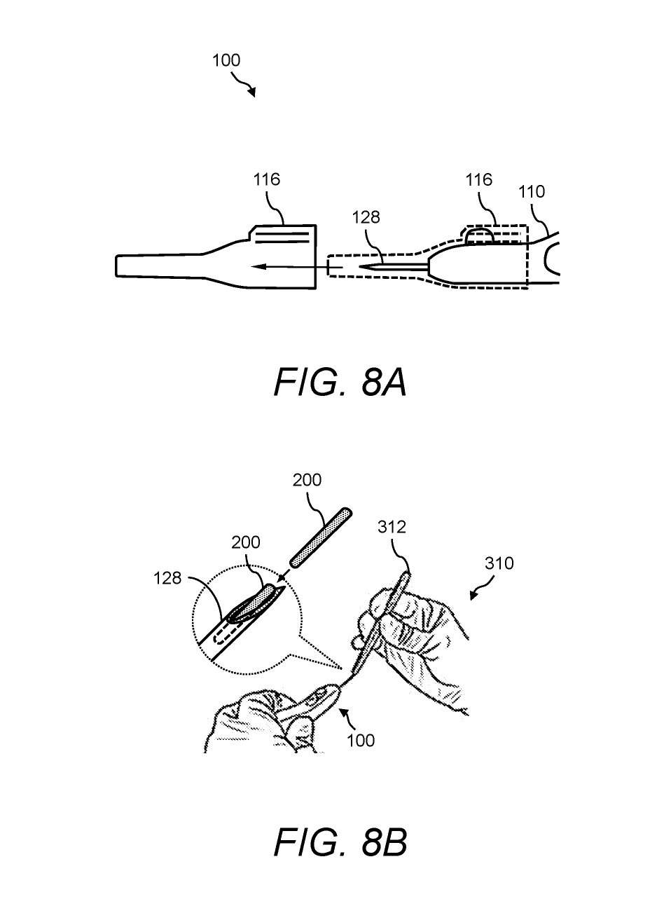

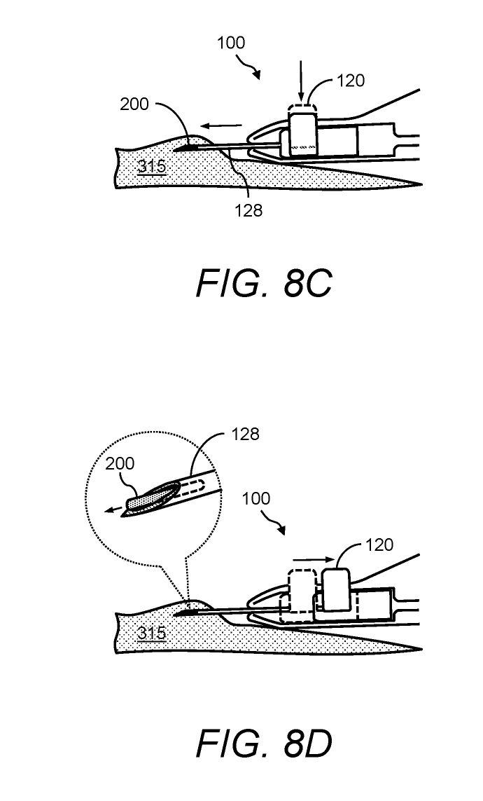

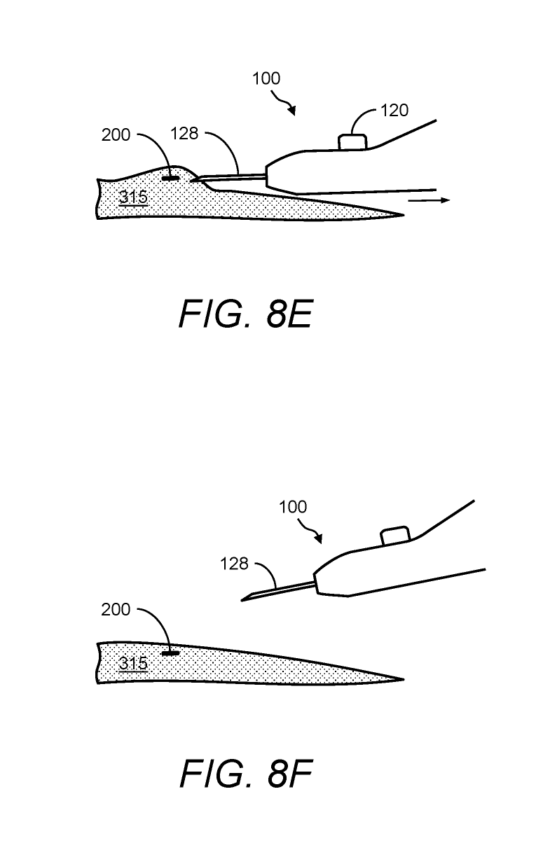

FIG. 8A through FIG. 8F illustrate a process of using inserter 100 to insert implant 200 under the skin, according to an embodiment. FIG. 8A shows the cap 116 being removed from the housing 110 of the inserter 100.

Next, the implant 200 may be loaded into the needle 128 by a user 310, such as a doctor, nurse, or any other medical practitioner. For example, FIG. 8B shows the user 310 using an instrument, such as tweezers 312, to load the implant 200 into the distal tip of the needle 128. In this step, the user 310 first advances the actuator 120, and thus the needle 128, fully forward and into an actuated and locked position (see FIG. 7A). Further, after loading the implant 200, the distal end of the wire 134 (see FIG. 7B) is abutted against the end of the implant 200 and acts as a stop. In other embodiments, the inserter 100 may be pre-loaded with implant 200. Similarly stated, in some embodiments, a user 310, such as a doctor, nurse, or other medical practitioner may not need to load implant 200 into the inserter 100. Instead, the inserter 100 may be provided to the user 310 as a product containing the implant 200.

Next, FIG. 8C shows the user 310 inserting the needle 128 into the skin 315 of the subject. The needle 128 functions similarly to a trocar and forms a channel in the body into which the implant 200 is intended to be placed. Namely, the needle 128 is inserted into the skin 315 and thereby forms a channel in the skin 315 and the neighboring body tissue.

The inserter 100 can be configured such that the needle 128 is inserted at an acute angle relative to the surface of the patient's skin 315. Specifically, as shown in FIG. 5B, the distal-most tip of the needle 128 (when the needle 128 is in the actuated configuration) and the lower surface of the housing 110 can define an angle 0 of less than 20 degrees, less than 10 degrees, or less than 5 degrees. Thus, when the needle is inserted (e.g., as shown in FIG. 8C), the bottom of the housing 110 can be placed on the surface of the subject's skin 315, and the needle 128 can be inserted at an acute angle to surface of the skin such that an appropriate shallow implantation (e.g., between 1 and 4 mm beneath the skin). Such an inserted can be superior to some known inserters, which are typically inserted at approximately 90 degrees relative to the surface of the skin. It can be difficult to target a specific depth using such a known inserter. Alternatively, some medical professionals may angle known inserters to attempt a more controlled shallow placement of an implant. Such a technique, however, is prone to error and inconsistency. Inserter 100, by having the distal-most tip of the needle 128 and the lower surface of the housing defining an acute angle, can allow safer, more reliable, and more repeatable positioning of the implant 200 at a desired depth.

Next, FIG. 8D shows the user retracting the needle 128 to release the implant 200 from the tip of the needle 128. Retracting the needle 128 can cause the needle to move between 0.5 and 4 mm. Retracting the needle 128 may not cause the needle to withdraw from the skin 315 of the patient. Similarly stated, a distance traveled by the needle 128 between the actuated configuration and the retracted configuration can be less than the depth at which the implant 200 is implanted. Moreover, even in the retracted configuration, the needle 128 remains exposed and at least partially extends from the housing 110.

Retracting the needle can be accomplished by the user pushing downward on the actuator 120, which unlocks the needle 128. Then, the user slides the actuator 120, and thus the needle 128, rearward. All the while, the wire 134 is held stationary against implant 200, and the needle 128 retracts relative to the implant 200. As a result, the implant 200 is released or ejected into the skin 315 as shown in FIG. 8E. That is, the needle 128 may be selectively removed, leaving behind the implant 200 in the channel created by the needle 128. Thus, retracting the needle 128 does not exert any force (other than minor frictional forces and associated normal forces) to the implant 200. Some embodiments described herein refer to ejecting an implant from a needle without applying a force to the implant. Such references should be understood as meaning no force, other than frictional forces caused by the needle sliding over the implant and associated normal forces, are applied directly to the implant. Next, the needle 128 is fully removed from the skin 315 as shown in FIG. 8F. The needle 128 can be removed from the skin 315 by withdrawing the inserter 100.

FIG. 9A through FIG. 9E illustrate a process of using the wire 134 of inserter 100 to assist deploying the implant 200 under the skin, according to an embodiment. Throughout the process, the distal end of the wire 134 is held abutted against (directly contacting) the proximal end of the implant 200, wherein the implant 200 is installed in the tip of the needle 128 and wherein the needle 128 is moveable separately from the wire 134.

For example, FIG. 9A shows the implant 200 positioned substantially within the tip of the needle 128. Next, FIG. 9B shows the implant 200 still held in place by the wire 134 and the needle slightly retracted relative to the wire 134 as compared with that shown in FIG. 9A and wherein a portion of the implant 200 now extends out of the needle 128 as compared with that shown in FIG. 9A. Next, FIG. 9C shows the implant 200 still held in place by the wire 134 and the needle slightly retracted relative to the wire 134 as compared with that shown in FIG. 9B and wherein a greater portion of the implant 200 now extends out of the needle 128 as compared with that shown in FIG. 9B. Next, FIG. 9D shows the implant 200 still held in place by the wire 134 and the needle slightly retracted relative to the wire 134 as compared with that shown in FIG. 9C and wherein a yet greater portion of the implant 200 now extends out of the needle 128 as compared with that shown in FIG. 9C. Next, FIG. 9E shows the implant 200 still held in place by the wire 134 and the needle retracted relative to the wire 134 as compared with that shown in FIG. 9D and wherein substantially the entirety of the implant 200 is out of the needle 128, i.e., the implant 200 is deployed.

In some embodiments, the needle 128 can move less than 5 mm, less than 10 mm, less than 20 mm, or any other suitable distance from the actuated to the retracted positions. The distance the needle travels between the actuated and retracted positions can be equal to or greater than a length of the implant. As discussed above, the implant 200 may be from about 0.1 mm to about 20 mm long in one example, from about 0.5 mm to about 10 mm long in another example, from about 1 mm to about 8 mm long in yet another example, or from about 2 mm to about 7 mm long in still another example. Thus, an embodiment in which the needle moves about 10 mm from the actuated position to the retracted position can be suitable to insert an implant 200 having a length of about 10 mm or less. In some embodiments, the length of the implant 200 can be about equal to the distance traveled by the needle 128 between the actuated and retracted positions.

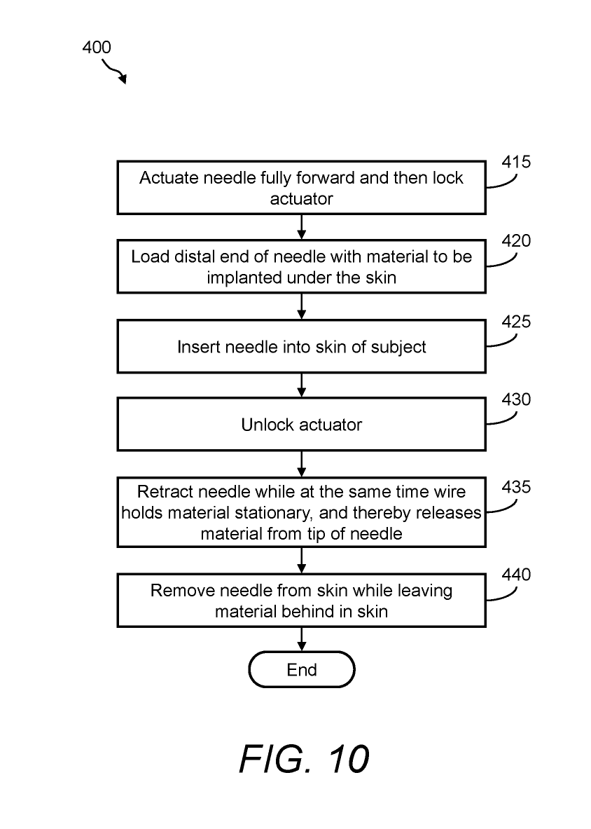

FIG. 10 is flow diagram of a method 400 of an inserter, such as inserter 100 shown and described above to insert an implant 200, such as a non-rigid implant, under the skin. For ease of discussion, the method 400 is described by reference to inserter 100 and/or implant 200, but it should be understood that method 400 may be carried out with any suitable inserter.

At 415, the needle is actuated fully forward and then the actuator is locked. For example, when the needle 128 is not preloaded with the implant 200, the user slides the actuator 120 fully forward and thereby actuates the needle 128 fully forward as shown, for example, in FIG. 7A and FIG. 8C. Then, the actuator 120 snaps into the locked position to hold the needle 128 in the fully forward (or actuated) position. For example, the user can release the actuator 120, which can cause the actuator 120 and the needle 128 to become locked into a fixed position relative to the housing 110.

At 420, the distal end of the needle is loaded with the implant to be implanted under the skin if not already preloaded. For example, the distal end of the needle 128 is loaded with the implant 200 to be implanted under the skin as shown, for example, in FIG. 7B and FIG. 8B. When the implant 200 is disposed within the needle, a proximal end of the implant is in direct contact with a distal end of the wire 134.

At 425, the needle is inserted into the skin of the subject. For example, the user inserts the needle 128 into the skin 315 of the subject as shown, for example, in FIG. 8C, by moving the entire inserter, with needle locked into the actuated position.

At 430, the actuator is unlocked. For example, the user pushes downward on the actuator 120, which unlocks the needle 128, as shown, for example, in FIG. 8C and FIG. 8D.

At 435, the needle 128 is retracted. Similarly stated the needle 128 can move proximally relative to the housing 110. For example and referring now to FIG. 8D, the user slides the actuator 120, and thus the needle 128, rearward. While the needle 128 is retracted, the wire 134 holds the implant 200 stationary (relative to the housing 110 and/or the patient), and accordingly the implant 200 is released from the tip of the needle 128. In this way, the implant 200 can be ejected from the needle 128 and into the patient's skin 315 without applying any force to the implant as shown in FIG. 8E and also according to the process shown in FIG. 9A through FIG. 9E.

At 440, the needle 128 is removed from the skin while leaving the implant 200 behind in the skin. For example, the implant 200 is released into the skin 315 as shown in FIG. 8E. That is, the needle 128 may be selectively removed, leaving behind the implant 200 in the channel created by the needle 128. The needle 128 can be removed from the skin by moving the entire inserter 100.

In other embodiments, the method 400 may further include moving the actuator 120 to remove the wire 134 after moving the actuator 120 to remove the needle 128. Further, in the method 400, the implant 200 may be placed in the subcutaneous space at from about 2 mm to about 6 mm from the skin surface.

The inserter 100 shown and described above is configured to be operated with a single hand. Similarly stated, at least events 425, 430, 435, and 440 can be performed one handed.

The disclosed inserter 100 and method 400 can be used to ensure reliable deployment of soft non-rigid materials, such as polymer- and/or hydrogel-based biosensors, under the skin of the subject. Additionally, the inserter 100 and the method 400 can be used to ensure reliable deployment of hard rigid materials under the skin of the subject.

The subject treated by the disclosed methods is desirably a human subject, although it is to be understood that the methods described herein are effective relative to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Some embodiments described herein include a wire. References to a wire should be understood to mean any structure thin enough to be disposed within a needle and rigid enough to maintain the position of the material or implant when the needle when the needle is retracted.

Although the foregoing subject matter has been described in some detail by way of illustration and example for purposes of clarity of understanding, it will be understood by those skilled in the art that certain changes and modifications can be practiced within the scope of the appended claims. Thus, while various embodiments have been described herein, it should be understood that they have been presented by way of example only, and not limitation.

Furthermore, although various embodiments have been described as having particular features and/or combinations of components, other embodiments are possible having a combination of any features and/or components from any of embodiments where appropriate as well as additional features and/or components. Where methods described herein indicate certain events occurring in certain order, the ordering of certain events may be modified. Additionally, certain of the events may be performed repeatedly, concurrently in a parallel process when possible, as well as performed sequentially as described above. Furthermore, certain embodiments may omit one or more described events.

What is claimed is:

1. An apparatus, comprising:
a housing;
a needle at least partially disposed within the housing and configured to be moved relative to the housing between an actuated configuration and a retracted configuration;
an actuator coupled to the needle and configured to move the needle between the actuated configuration and the retracted configuration; and
a wire at least partially disposed within the needle, the wire immovably coupled to the housing.

2. The apparatus of claim 1, further comprising a biosensor disposed within the needle.

3. The apparatus of claim 1, further comprising a biosensor disposed within the needle such that a proximal end of the biosensor directly contacts a distal end of the wire.

3. The apparatus of claim 1, further comprising a non-rigid biosensor.

4. The apparatus of claim 1, further comprising a non-rigid biosensor disposed within the needle such that a proximal end of the biosensor directly contacts a distal end of the wire, the non-rigid biosensor being compressible and having a porous structure that is subject to damage by compression.

5. The apparatus of claim 1, further comprising a hydrogel implant disposed within the needle.

6. The apparatus of claim 1, further comprising a hydrogel implant disposed within the needle, the actuator configured to eject the hydrogel implant from the needle without applying a force to the hydrogel implant.

7. The apparatus of claim 1, further comprising an actuator spring configured to urge the actuator into a locked position, the actuator and the housing collectively configured to prevent the needle from moving between the actuated configuration and the retracted configuration when the actuator is in the locked position.

8. The apparatus of claim 1, wherein the actuator is configured to move between a locked configuration and an unlocked configuration when the needle is in the actuated configuration, in the locked configuration, the actuator and the housing collectively configured to prevent the needle from moving to the retracted position, in the unlocked configuration, the actuator configured to move the needle from the actuated configuration to the retracted configuration.

9. The apparatus of claim 1, wherein the needle is configured to be moved between the actuated configuration and the retracted configuration with one hand.

10. An apparatus, comprising:
a housing having a bottom surface;
a needle at least partially disposed within the housing, the needle having a distal tip, the distal tip of the needle and the bottom surface of the housing forming an angle of less than 10 degrees; and
an implant disposed within the needle, the needle configured to insert the implant under skin of the patient, the bottom surface of the housing configured to be placed in contact with the skin of the patient while the implant is being inserted such that the needle is inserted into the skin at an angle of less than 10 degrees.

11. The apparatus of claim 10, wherein the implant is a biosensor.

12. The apparatus of claim 10, wherein the needle is configured to be moved relative to the housing between an actuated configuration and a retracted configuration, the needle in the actuated configuration when the distal tip of the needle and the bottom surface of the housing form the angle of less of than 10 degrees.

13. The apparatus of claim 10, further comprising a wire at least partially disposed within the needle, the wire immovably coupled to the housing, a distal end of the wire contacting a proximal end of the implant, the needle configured to move relative to the housing.

14. The apparatus of claim 10, wherein the implant is non-rigid.

15. A method, comprising:
moving body of an inserter having a needle such that the needle is inserted into a patient, the needle containing an implant;
moving the needle relative to a body of the inserter such that implant is ejected from the needle; and
moving the body of the inserter such that the needle is withdrawn from the patient leaving the implant within the patient.

16. The method of claim 15, further comprising:
locking the needle into an actuated configuration prior to inserting the needle into the patient;
unlocking the needle after inserting the needle into the patient and before moving the needle, the needle moved from the actuated configuration to a retracted configuration.

17. The method of claim 15, wherein moving the needle relative to the body of the inserter does not cause the needle to be withdrawn from the patient.

18. The method of claim 15, wherein moving a portion of the needle protruding from the housing when the needle inserted into a patient is at least 10 mm long and the needle is moved less than 5 mm relative to the body of the inserter.

19. The method of claim 15, wherein the inserter includes a wire partially disposed within the needle that remains stationary relative to the body of the inserter while the needle is moved relative to the body of the inserter, the wire contacting the implant.
